(19) United States
(12) Patent Application Publication
(10) Pub. No.: US 2011/0257513 A1
(43) Pub. Date: Oct. 20, 2011

(54) IMMOBILIZATION APPARATUS FOR MEDICAL IMAGING

(75) Inventors: Steven Toddes, Westminster, MA (US); Mathew Brevard, Worcester, MA (US); A. Robert Sohval, Ridgewood, NJ (US)

(73) Assignee: MICROMRI, INC., Langhorne, PA (US)

(21) Appl. No.: 13/130,868

(22) PCT Filed: Nov. 23, 2009
(86) PCT No.: PCT/US09/65474

§ 371 Date: May 24, 2011

Related U.S. Application Data
(60) Provisional application No. 61/117,341, filed on Nov. 24, 2008.

(51) Int. Cl. A61B 5/055

(52) U.S. Cl. 600/415

(57) ABSTRACT

An apparatus for immobilizing an anatomical feature during a magnetic resonance imaging process of the anatomical feature is disclosed. The apparatus has a housing, which defines a central opening to accommodate the anatomical feature. The apparatus further includes a coil member fixedly mounted in the housing. The coil member processes an RF signal. The apparatus further includes a first support member disposed at a side of the housing and a second support member disposed at an opposite side of the housing. The first and second support members are fixed with respect to the housing.

The present invention relates generally to medical imaging systems for medical treatment and/or diagnostic purposes. More particularly, the present invention relates to an apparatus for immobilizing an anatomical feature, especially a wrist, during medical imaging and image analysis.

Osteoporosis is a bone disease that affects a large portion of the population, especially post-menopausal women, which leads to increased risk of fracture. The disease is characterized by decreased bone mineral density and deteriorated bone microarchitecture. Clinical research studies have shown that bone microarchitecture makes a significant contribution to fracture risk and that changes in bone microarchitecture in response to therapeutic treatment can be detected much earlier than changes in bone mineral density. While commercially available x-ray devices can monitor changes in bone mineral density, non-invasive imaging devices to quantify bone microarchitecture would be very useful.

Characterization of bone microarchitecture requires visualization and quantification of the network of trabecular bone such as found at the end of the long bone in the wrist. The imaging system must be capable of acquiring volumetric data sets with 3D voxel size on the order of 100 microns, the same scale size as trabecular bone structure.

Magnetic Resonance Imaging (MRI) has been shown to be capable of achieving this resolution when imaging appendages such as the wrist. A special pulse sequence and image processing algorithm are required for data acquisition and data processing, respectively. The scan time to acquire a 3D volumetric set of data is in the range of 5-15 minutes.

To create an MR image of the wrist, an RF (Radio Frequency) coil tuned according to the field strength of the magnet must be placed in close proximity to the wrist.

During the image scanning process, movement of the wrist must be minimized to prevent blurring and other motion-related image artifacts, which degrade image quality and measurement accuracy. Since the voxel size is so small, even a small amount of patient movement can degrade a scan or necessitate a study to be repeated.

However, due to the inherent flexibility of the wrist joint that couples the hand to the arm, it is difficult to immobilize the wrist itself. While a close-fitting wrist coil is desirable for generating high quality MRI images, attempts to immobilize the joint, for example, by inserting pads between the wrist and the inner wall of the coil housing have been found to be inadequate. Efforts to use a separate immobilization device, such as a vacuum fixation bag, have failed to provide consistent and adequate immobilization. In addition, exam set up and patient positioning using such a device are cumbersome and time consuming.

Furthermore, under many circumstances, serial exams for the same patient with respect to the same anatomical feature (such as the wrist) are important for monitoring disease regression or progression during the course of treatment, which may run over the course of months or years. Accordingly, precise and consistent registration at different time between the wrist and the RF coil as well as between the RF coil and the MR scanner is critical for studying and controlling the treatment. However, for the known systems, each time when the equipment is set up to scan the wrist, the resultant image may not be consistent with the previous images in terms of angle and orientation.

Thus, it is desirable to provide an apparatus for immobilizing and stabilizing the wrist during a medical imaging process, so as to obtain a high quality, high-resolution volumetric image of the wrist.

Thus, it is also desirable to provide an apparatus for precisely and consistently registering the wrist, RF coil and MR scanner with one another during a medical imaging process, so as to ensure that serial exams on the same patient are recorded at precisely the same anatomic location even if the exams are performed at different time (for, example, months or years apart).

Thus, it is also desirable to provide an apparatus for maximizing patient comfort during the exam with medical imaging, which further reduces the likelihood of voluntary or involuntary patient movement during the scan.

Thus, it is also desirable to provide an apparatus for streamlining the patient setup procedure during a medical imaging process in order to minimize the total time required to set up and perform the exam.

### SUMMARY OF THE INVENTION

As described herein, the various exemplary embodiments of the present invention overcome one or more of the above or other disadvantages known in the art.

An aspect of the present invention relates to an apparatus for immobilizing an anatomical feature during a medical imaging process of the anatomical feature. The apparatus includes a housing, which has a central opening to accommodate the anatomical feature. The apparatus further includes a coil member fixedly mounted in the housing. The coil member processes an RF signal. The apparatus further includes a first support member disposed at a side of the housing and a second support member disposed at an opposite side of the housing. The first and second support members are fixed with respect to the housing.

Another aspect of the present invention relates to a system for performing an MRI imaging of an anatomical feature of a patient. The system includes an apparatus for immobilizing the anatomical feature during the MRI imaging. The apparatus includes a housing, which includes a central opening configured to accommodate the anatomical feature. The apparatus includes a coil member fixedly mounted in the housing, the coil member processing an RF signal. The apparatus further includes a first support member disposed at a side of the housing and a second support member disposed at an opposite side of the housing. The apparatus further includes a base member. The housing, the first support member and the second support member are fixedly mounted on a common side of the base member. The system further includes an MR scanner. The scanner includes, inter alia, a table for supporting the patient. The system further includes a means for releasably locking the base member of the apparatus to the table of the MR scanner.

### BRIEF DESCRIPTION OF THE DRAWINGS

FIG. 1 is a perspective view of an apparatus for immobilizing a wrist during medical imaging, according to an exemplary embodiment of the present invention;

FIG. 2 is a partial perspective view of the apparatus in FIG. 1, also showing a forearm, a wrist and a hand placed in position for medical scanning;

FIG. 3 is a partial perspective view of the apparatus in FIG. 2, with some components of the apparatus removed to better depict the details of the apparatus; and

FIG. 4 is a schematic block view of an MRI imaging system according to an exemplary embodiment of another aspect of the present invention.

### DETAILED DESCRIPTION OF THE EXEMPLARY EMBODIMENTS OF THE INVENTION

It is contemplated that the teaching of the description set forth below is applicable to all types of medical imaging systems, including but not limited to MRI systems. The present invention is therefore not intended to be limited to any particular MRI apparatus or configuration described in the exemplary embodiments of the present invention.

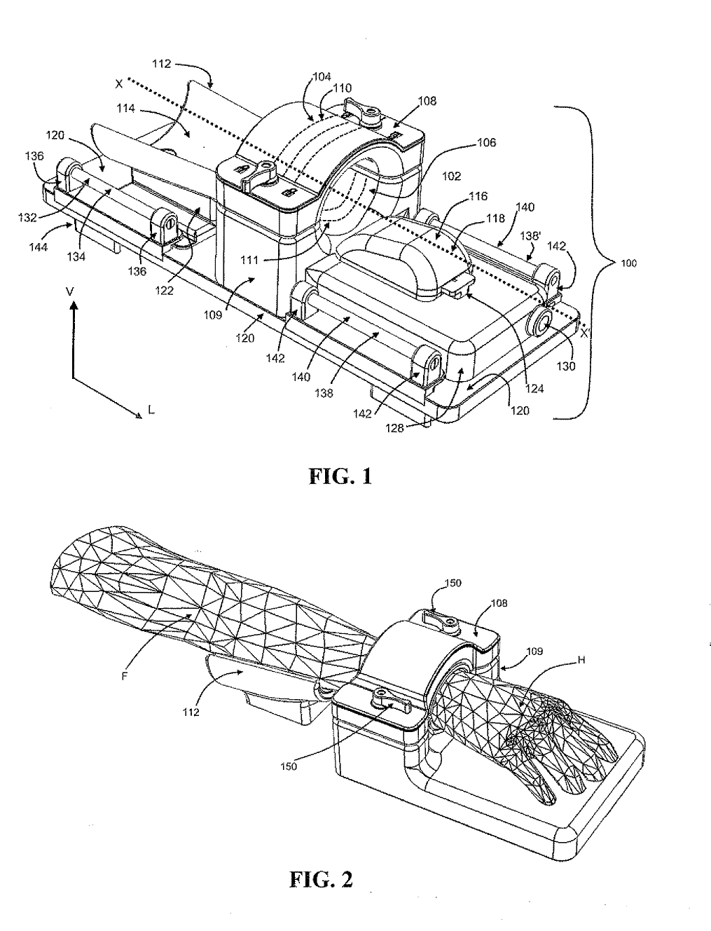

FIG. 1 illustrates a perspective view of an apparatus 100 for immobilizing an anatomical feature during medical imaging, according to an exemplary embodiment of the present invention. A vertical direction is defined as V and a longitudinal direction is defined as L, as shown in FIG. 1.

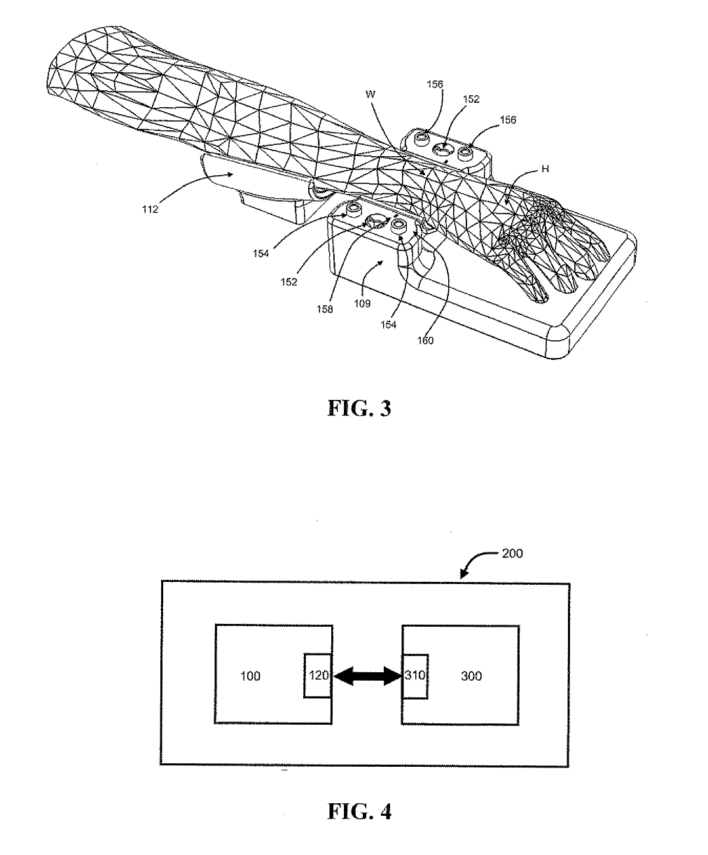

As shown, the apparatus 100 includes a housing 102 and coil member 104 (shown in dashed lines) fixedly mounted in the housing 102. The coil member 104 processes an RF signal, for example, in response to a magnetic filed generated during medical imaging. The coil member 104 can be a receive-only coil or a transmit/receive coil. The housing 102 defines a central opening 106 for accommodating an anatomical feature, such as a wrist W, as shown in FIGS. 2 and 3.

The housing 102 includes an upper portion 108 and an opposite lower portion 109, releasably fixable to each other through a locking means, which will be described later with reference to FIGS. 2 and 3. The central opening 106 is defined by the connection of the upper portion 108 and the lower portion 109 with each other.

The coil member 104 includes an upper portion 110 fixed in the upper portion 108 of the housing 102, and a lower portion 111 fixed in the lower portion 109 of the housing 102.

The apparatus 100 further includes a first support member 112 disposed at one side of the housing 102. The first support member 112 is adapted to support a forearm F associated with the wrist W, as best seen in FIGS. 2 and 3. The first support member 112 is fixed with respect to the housing 102, thereby immobilizing the forearm F with respect to the wrist W. In the shown embodiment, the first support member 112 assumes the shape of a concave plate. The first support member 112 has a curved surface 114, which is substantially conically shaped. The curved surface 114 facilitates the first support member 112 to better conform to the natural profile of the forearm F.

The apparatus 100 further includes a second support member 116 disposed at another side of the housing 102, longitudinally opposite to the first support member 112. The second support member 116 is adapted to support a hand H associated with the wrist W, which is shown in FIGS. 2 and 3. The second support member 116 is fixed with respect to the housing 102, thereby immobilizing the hand H with respect to the wrist W. The second support member 116 includes a curved surface 118, which is convex to allow the palm of the hand to rest thereon.

The housing 102, the first support member 112 and the second support member 116 are constructed to substantially aligned along an axis X-X′, which can be defined with respect to the wrist W, the forearm F and the hand H.

The apparatus 100 further includes a base member 120, on which the housing 102, the first support member 112 and the second support member 116 are fixedly mounted. For example, the base member 120 can be a piece of solid material, such as a metal or plastic plate. The base member 120 provides a common structure for mounting the housing 102, the first support member 112 and the second support member 116, thereby providing a structurally integral apparatus for immobilizing the forearm F, the wrist W and the hand H. In addition, the common base member 120 provides positional rigidity between the housing 102 and the first support member 112 and between the housing 102 and the second support member 116. Thus, for each time the wrist W is being scanned, the wrist W can be maintained at positions satisfactorily consistent with one another.

The first support member 112 can be lined with a removable pad (not shown) to provide additional patient comfort or to better support smaller arms. A pedestal 122 can be provided between the first support member 112 and the base member 120, for adjusting the vertical position of the first support member 112 with respect to the central opening 106, which would allow a patient to place his/her forearm and wrist in a comfortable position. Accordingly, undesirable movement of the forearm and wrist due to the patient's uncomfortable feeling can be avoided. The first support member 112 can be attached to the pedestal 122, which is in turn fixed to the base member 120. For example, the pedestal 122 can match the height of the first support member 112 nearest to the coil member 104 to the height of the bottom of the central opening 106 in the lower portion 109 of the housing 102. Optionally, the longitudinal location of the pedestal 122 can be adjusted along the axis X-X′, to accommodate different lengths of the arms. Optionally, a locking mechanism, such as a ratchet, can be used to fix the location of the pedestal 122 after it has been placed in a desired position, to eventually fix the pedestal 122 as well as the first support member 112 with respect to the base member 120.

A rail 124 can be provided for slidably adjusting the longitudinal position of the second support member 116 with respect to the central opening 106, which would allow a patient to place his/her wrist and hand in a comfortable position. Accordingly, undesirable movement of the hand and wrist due to the patient's uncomfortable feeling can be avoided. A locking mechanism, such as a ratchet, can be provided to fix the second support member 116 with respect to the rail 124.

An insulated case 128 can be further provided between the second support member 116 and the base member 120. The second support member 116 is attached on the insulated case 128. The case 128 may optionally receive electronic components such as preamps (not shown) for processing signals from the coil member 104. The electric cable (not shown) carrying signals between the coil member 104 and an external MR scanner (as shown schematically in FIG. 4, which will be described later) exits from the case 128 at a port 130. The port 130 is disposed at a position away from the fingers, to avoid undesirable interfering with patient setup. A connector (not shown) on the end of the cable mates with a corresponding connector (not shown) on the MR scanner.

The apparatus 100 further includes a first pair of extensions 132 and 132′ (not shown), associated with the first support member 112. The first pair of extensions 132 and 132′ are disposed at either side of the first support member 112, for applying a cloth strap around the forearm F to avoid movement of the forearm F. For example, the first pair of extensions 132 and 132′ each include a longitudinally extending rail 134 attached to struts 136 vertically extending from the base member 120. In this way, the cloth strap looping around the rails may be drawn firmly over the top of the patient's forearm to prevent movement of the forearm during a scan. Optionally, the cloth strap can be provided with Velcro® surfaces.

The apparatus 100 further includes a second pair of extensions 138 and 138′, associated with the second support member 116. The second pair of extensions 138 and 138′ are disposed at either side of the second support member 116, for applying a cloth strap around the hand H to avoid movement of the hand H. For example, the second pair of extensions 138 and 138′ each include a longitudinally extending rail 140 attached to struts 142 vertically extending from the base member 120. In this way, the cloth strap looping around the rails may be drawn firmly over the top of the patient's hand to prevent movement of the hand during a scan. Optionally, the cloth strap can be provided with Velcro® surfaces. The cooperation of the second support member 116 and the cloth strap on top of the hand provides robust mechanical registration and significantly restricts both translational and rotational movement of the patient's wrist with respect to the axis X-X′.

The base plate 120 is firmed attached a patient table (as shown schematically in FIG. 4, which will be described later) of the external MR scanner, for example, through plastic teeth 144 inserted in the base plate 120. The plastic teeth 144 engage corresponding elongated slots along the side of the patient table. Alternatively, the base plate can be mounted to a curved plastic form (not shown), which matches the curvature of the patient table. When the patient lies on curved plastic form, the location of the wrist coil is secured.

100351 Using a mechanical means to repeatably fix the position of the base plate 120 with respect to the isocenter of the MR scanner ensures, that any spatial non-uniformity, in the magnetic field strength away from isocenter that might influence image quality or affect quantitative outputs of the post-processing software, is repeatable from scan to scan.

Except for the cable port 130, the coil member 104, the housing 102, the first support member 112 and the second support member 116 are disposed to be symmetric about the center line X-X′, which enables the same system to be used for imaging either the left or right wrist.

For the purpose of clarity of use, FIG. 2 presents the coil member 104, the housing 102 and the first support member 112 together with a schematic representation of the forearm F properly positioned for imaging.

FIG. 3 is a partial perspective view of the apparatus 100, with some components removed to better depict the details of the apparatus. Now referring to both FIGS. 2 and 3, the upper portion 108 of the housing 102 (holding the upper portion 110 of the coil member 104) and the lower portion of the housing 102 (holding the lower portion 111 of the coil member 104) can be releasably coupled to one another through a locking means. Thus, during operation, an MR technician first detaches the upper portion 108 from the lower portion 109, to allow the patient to comfortably place his/her forearm, wrist and hand in position. Subsequently, after ensuring the forearm, wrist and hand are in right positions, the technician locks the upper portion 108 and the lower portion 109. Accordingly, the upper portion 108 and the lower portion 109 are mechanically fixed with each other to avoid movement of the forearm, hand and wrists; and the electrical connection between the upper potion 110 and the lower portion 111 of the coil member 104 is simultaneously established, to active the coil member 104.

In the shown embodiment, the locking means includes a pair of levers 150 and a pair of receptacles 152, the levers engaging the receptacles, respectively. The levers 150 are disposed on one of the upper portion 108 and lower portion 109; and the receptacles 152 are disposed on the other of the upper portion 108 and the lower portion 109. FIG. 3 also illustrates a first pair of electrical outlets 154 and a second pair of electrical outlets 156, disposed at either side of the wrist W. The electrical outlets 154 and 156 are electrically connected to the lower portion 111 of the coil member 104. When the upper portion 108 and the lower portion 109 of the housing 102 are mechanically connected to one another, pins (not shown) electrically connected to the upper portion 110 of the coil member 104 are inserted into the outlets 154 and 156, to complete the electrical circuit for the coil member 104.

In order to assist the MR technical to accurately examine the position of the patient's wrist, a reference marker 158 is further provided. For example, the reference marker 158 can be a fiducial reference marker provided on an upper surface 160 of the lower portion 109 of the housing 102, as shown in. FIG. 3.

When the upper portion 108 of the housing 102 is removed, the reference marker 158 is exposed, adjacent to the center of the exposed upper surface 160 of the lower portion 109.

In operation, the forearm F rests in the first support member 112 and the wrist W rests in the lower portion 109 of the housing 102. The technician ensures that the patient's ulnar styloid process (the bony protuberance on the upper-outside part of the wrist, not indicated) is positioned next to the fiducial reference marker 158. Thus, every time when the upper portion 108 of the housing 102 is removed, the reference marker 158 is revealed to assist the technician to properly position the patient's wrist as well as forearm and hand.

FIG. 4 is a schematic block view of an MRI imaging system 200 according to an exemplary embodiment of another aspect of the present invention. The system 200 includes the apparatus 100 for immobilizing the patient's wrist, forearm and hand, and an MR scanner 300. The MR scanner 300, for example, can be any type of known MR scanner for performing various functions, including translating the patient to acquire a plurality image frames, maintaining a magnetic field at a desirable level and so on. The MR scanner 300 includes a patient table 310, on which the patient lies. The immobilizing apparatus 100 is mounted to the table 310 in a fixed manner, prior to the scanning and imaging process. Specifically, the base member 120 of the apparatus 100 is mounted to the table 310, for example, through the engagement between the teeth 144 of the apparatus 100 and a groove formed in the table 310. Of course, any other suitable coupling means can be used without deviating from the scheme of the invention.

For the MRI imaging system 200, the apparatus 100 immobilizes the patient's wrist to allow the scanner 300 and coil member 104, in cooperation, to acquire high quality images for diagnostic and treatment purposes.

In the system 200, the positioning of the wrist only relies on the alignment of the patient's ulnar styloid process with the fiducial marking, which ensures consistent and repeatable positioning of the wrist anatomy.

Only by securing the patient's arm and hand using fixtures that are rigidly attached to the coil, is mechanical registration achieved and even small motion of the wrist substantially eliminated. Integration of the immobilization apparatus with the coil into a single mechanical structure maintains proper and repeatable registration of the wrist to the coil, significantly attenuates patient potion, and ensures high image quality and measurement accuracy.

While there have shown and described and pointed out fundamental novel features of the invention as applied to various specific embodiments thereof, it will be understood that various omissions and substitutions and changes in the form and details of the apparatus illustrated, and in their operation, may be made by those skilled in the art without departing from the spirit of the invention. For example, it is expressly intended that all combinations of those elements which perform substantially the same function in substantially the same way to achieve the same results are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A apparatus for immobilizing an anatomical feature during a magnetic resonance imaging process of the anatomical feature, comprising:
a housing comprising a central opening configured to accommodate the anatomical feature;
a coil member fixedly mounted in the housing, the coil member processing an RF signal; and
a first support member disposed at a side of said housing and a second support member disposed at an opposite side of said housing, said first and second support members fixed with respect to said housing.

2. The apparatus according to claim 1, further comprising a base member, said housing, said first support member and said second support member fixedly mounted on the base member.

3. The apparatus according to claim 1, wherein said housing, said first support member and said second support member are aligned substantially along an axis defined with respect to the anatomical feature.

4. The apparatus according to claim 1, wherein said central opening is configured to accommodate a wrist, said first support member is configured to support a forearm associated with the wrist, and said second support member is configured to support a hand associated with the wrist.

5. The apparatus according to claim 1, wherein said housing comprises an upper portion and a lower portion, said upper portion and said lower portion releasably connectable to each other through a locking means.

6. The apparatus according to claim 5, wherein said locking means comprises: at least one lever provided in one of said upper portion and said lower portion; and at least one receptacle provided in the other of said upper portion and said lower portion, said lever configured to engage said receptacle.

7. The apparatus according to claim 5, wherein said coil member comprises: an upper portion fixed in said upper portion of said housing; and a lower portion fixed in said lower portion of said housing.

8. The apparatus according to claim 7, wherein said upper portion of said coil member comprises one of a pin and an outlet, and said lower portion of said coil member comprises the other of a pin and an outlet, said pin and said outlet engaging each other to establish an electrical connection, when said upper portion of said housing and said lower portion of said housing are connected to each other.

9. The apparatus according to claim 2, further comprising a pedestal disposed between said first support member and said based member, for adjusting the vertical position of said first support member with respect to said central opening.

10. The apparatus according to claim 1, further comprising a rail for adjusting the longitudinal position of said second support member with respect to said central opening.

11. The apparatus according to claim 2, further comprising an insulated case disposed between said second support member and said base member, for accommodating an electric cable.

12. The apparatus according to claim 11, wherein said insulated case comprises a port, through which said electric cable exits.

13. The apparatus according to claim 4, wherein said first support member comprises a substantially curved surface configured to support the forearm.

14. The apparatus according to claim 13, wherein said substantially curved surface comprises a substantially conically shaped concave surface.

15. The apparatus according to claim 4, wherein said second support member comprises a substantially curved surface configured to support the palm of the hand.

16. The apparatus according to claim 15, wherein said substantially curved surface comprises a convex surface.

17. The apparatus according to claim 4, further comprising a first pair of extensions connected to said base member, said first pair of extensions disposed at either side of said first support member, for applying at least one strap on the forearm.

18. The apparatus according to claim 4, further comprising a second pair of extensions connected to said base member, said second pair of extensions disposed at either side of said second support member, for applying at least one strap on the hand.

19. The apparatus according to claim 5, further comprising at least one reference marker provided on a surface of said lower portion of said housing.

20. The apparatus according to claim 2, wherein said base member comprises at least one tooth for coupling said base member to an external MR scanner.

21. A system for performing MRI imaging of an anatomical feature of a patient, comprising:
an apparatus for immobilizing the anatomical feature during the MRI imaging, said apparatus comprising:
a housing comprising a central opening configured to accommodate the anatomical feature,
a coil member fixedly mounted in the housing, the coil member processing an RF signal,
a first support member disposed at a side of said housing and a second support member disposed at an opposite side of said housing, and
a base member, said housing, said first support member and said second support member fixedly mounted on a common side of said base member;
an MR scanner comprising a table for supporting the patient; and
a means for releasably locking said base member of said apparatus to said table of said MR scanner.
